(19) United States
(12) Patent Application Publication
(10) Pub. No.: US 2025/0155060 A1
(43) Pub. Date: May 15, 2025

(54) TUBE CONNECTION SYSTEM AND TUBE CONNECTOR SET

(71) Applicant: Sanplatec Corporation Ltd., Osaka (JP)

(72) Inventors: Satoshi KATOU, Osaka (JP); Junichi KUWABARA, Osaka (JP)

(21) Appl. No.: 19/028,325

(22) Filed: Jan. 17, 2025

Related U.S. Application Data
(63) Continuation of application No. PCT/JP2023/036776, filed on Oct. 10, 2023.

(51) Int. Cl. F16L 29/00

(52) U.S. Cl. CPC F16L 29/007

(57) ABSTRACT

A tube connection system includes a holder holding first and second fluid-path members, a heater, and an extractor. Each fluid-path member includes an internal channel along a first direction, a fluid path end at an end of the internal channel and capable of being fusion-bonded, and a sealing element of higher thermal conductivity than that of the fluid path end for covering the fluid path end. The holder holds the first and second fluid-path members with respective sealing elements facing and being pressed against each other. The heater heats the sealing element of each fluid-path member. The extractor pulls out the sealing element of each fluid-path member in a second direction orthogonal to the first direction.

### TECHNICAL FIELD

The present disclosure relates to a tube connection system for aseptically connecting tubes, and to a tube connector set.

### BACKGROUND ART

Devices for aseptically joining tubes are conventionally known. For example, Japanese Patent No. 4262249 discloses a tube joining device for joining two tubes. The conventional tube jointing device disclosed in the above document includes a pair of clamps that hold two tubes at two points with a gap between them, a cutting plate that cuts the two tubes, and a means for moving the clamps as appropriate. When joining tubes, first, the two tubes are cut between a pair of clamps using a heated cutting plate, and the clamps are rotated such that the cut surfaces of the two tubes face each other. Next, the cutting plate is retreated and the hot cutting surfaces are pressed against each other in the axial direction, causing the cutting surfaces to fuse to form a single tube. The above tube joining device is used to join tubes aseptically, for example, when replacing a dialysis fluid bag and a waste fluid bag.

The conventional tube joining device described above requires precise and appropriate movement of the clamps, which has led to an increase in the complexity and size of the overall device. In addition, if the diameters or materials of the two tubes to be joined are different, the tubes can not be joined properly.

### SUMMARY OF THE INVENTION

The present disclosure has been considered in light of the above circumstances, and an object of the present disclosure is to provide a tube connection system suitable for aseptically connecting various tubes.

To solve the above issues, the present disclosure may adopt the following technical measures.

A tube connection system provided according to a first aspect of the present disclosure includes a holder that holds a first fluid-path member and a second fluid-path member, a heater, and an extractor. Each of the first fluid-path member and the second fluid-path member includes an internal channel penetrating in a first direction, a fluid path end located at an end of the internal channel and capable of being fusion-bonded, and a sealing element having a higher thermal conductivity than that of the fluid path end and covering the fluid path end. The holder is capable of holding the first fluid-path member and the second fluid-path member with the respective sealing elements facing and being pressed against each other. The heater is capable of heating the sealing element of each of the first fluid-path member and the second fluid-path member. The extractor is capable of pulling out the sealing element of each of the first fluid-path member and the second fluid-path member in a second direction orthogonal to the first direction.

In a preferred embodiment, the tube connection system further includes the first fluid-path member and the second fluid-path member.

In a preferred embodiment, the first fluid-path member includes a first tube connector, the second fluid-path member includes a second tube connector, and each of the first tube connector and the second tube connector includes a tube attachment portion disposed on a first side in the first direction, the fluid path end disposed on a second side in the first direction, and the internal channel penetrating from the tube attachment portion to the fluid path end in the first direction.

In a preferred embodiment, the holder includes a first joint frame that holds the first tube connector and a second joint frame that holds the second tube connector, and when the first joint frame and the second joint frame are fitted together and integrated, the first joint frame and the second joint frame are capable of holding the first tube connector and the second tube connector with the sealing elements of the first fluid-path member and the second fluid-path member facing and being pressed against each other.

In a preferred embodiment, at least one of the first joint frame and the second joint frame includes an elastic function portion, and the elastic function portion causes the sealing elements of the first fluid-path member and the second fluid-path member to be pressed against each other.

In a preferred embodiment, at least one of the first tube connector and the second tube connector includes an integral or separate elastic function portion, and the elastic function portion causes the sealing elements of the first fluid-path member and the second fluid-path member to be pressed against each other.

In a preferred embodiment, the extractor is configured to fix the first joint frame and the second joint frame in a fitted position, grip the sealing elements with a seal clamping member capable of heating the sealing elements, and then pull out the sealing elements by moving the seal clamping member in the second direction.

In a preferred embodiment, the extractor is configured to grip the sealing elements with a seal clamping member capable of heating the sealing elements in a state where the first joint frame and the second joint frame are fitted together, and then pull out the sealing elements by a mechanism that pushes off at least one of the first joint frame and the second joint frame.

In a preferred embodiment, the fluid path end includes an annular first end surface facing the second side in the first direction and a fluid path end recess that is recessed from the first end surface toward the first side in the first direction on a radially inner side of the first end surface, and the fluid path end recess surrounds the internal channel as viewed in the first direction.

In a preferred embodiment, each of the first tube connector and the second tube connector includes an annular elastic element attached to the fluid path end recess and having a higher heat resistant temperature than that of the first end surface, the annular elastic element includes a first through-hole communicating with the internal channel and a second end surface located on the second side in the first direction from the first end surface in a natural state and facing the second side in the first direction, and the sealing element covers the first end surface and the second end surface.

In a preferred embodiment, each of the first tube connector and the second tube connector includes an annular element attached to the fluid path end recess and having a higher heat resistant temperature than that of the first end surface, the annular element includes a second through-hole communicating with the internal channel and a third end surface facing the second side in the first direction, and the sealing element covers the first end surface and the third end surface.

In a preferred embodiment, the sealing element includes a thin metal plate or a thin metal film.

In a preferred embodiment, the sealing element includes a first portion overlapping with the fluid path end as viewed in the first direction, and a second portion extending out from the first portion in a direction orthogonal to the first direction.

In a preferred embodiment, the second portion is provided with an anti-slip structure on a first surface thereof that faces the first side in the first direction.

In a preferred embodiment, each of the first fluid-path member and the second fluid-path member includes a tube stopper that is attachable in such a manner that a tube fitted over the tube attachment portion is interposed between the tube stopper and the tube attachment portion.

A tube connector set provided according to a second aspect of the present disclosure is a tube connector set that constitutes the tube connection system according to the first aspect of the present disclosure and includes the first fluid-path member and the second fluid-path member.

Other features and advantages of the present disclosure will become apparent from the detailed description given below with reference to the accompanying drawings.

### BRIEF DESCRIPTION OF THE DRAWINGS

FIG. 1 is a schematic front view of a tube connection system according to an embodiment of the present disclosure.

FIG. 2 is a perspective view of a first fluid-path member and a first joint frame.

FIG. 3 is a sectional view taken along line III-III in FIG. 2.

FIG. 4 is a sectional view taken along line IV-IV in FIG. 3.

FIG. 5 is a perspective view of a second fluid-path member and a second joint frame.

FIG. 6 is a sectional view taken along line VI-VI in FIG. 5.

FIG. 7 is a sectional view taken along line VII-VII in FIG. 6.

FIG. 8 is a perspective view showing the first and the second joint frames fitted together and integrated.

FIG. 9 is a sectional view taken along line IX-IX in FIG. 8.

FIG. 10 is a sectional view showing the fitted portion of the first and the second joint frames.

FIG. 11 is a sectional view showing the first and the second tube connectors connected together.

FIG. 12 is a sectional view showing a first variation of the first and the second fluid-path members.

FIG. 13 is a sectional view showing the first and the second fluid-path members according to the first variation connected together.

FIG. 14 is a sectional view showing a second variation of the first and the second fluid-path members.

FIG. 15 is a sectional view showing the first and the second fluid-path members according to the second variation connected together.

FIG. 16 is a sectional view showing the first and the second fluid-path members according to a third variation connected together.

FIG. 17 is a sectional view showing the first and the second fluid-path members according to a fourth variation connected together.

FIG. 18 is a sectional view showing the first and the second fluid-path members according to a fifth variation connected together.

FIG. 19 is a schematic front view of a tube connection system according to another embodiment of the present disclosure.

FIG. 20 is a front view of a connection apparatus.

FIG. 21 is a schematic diagram showing an example of a tube connector set according to the present disclosure.

### DETAILED DESCRIPTION OF EMBODIMENTS

The following describes preferred embodiments of the present disclosure in detail with reference to the drawings.

In the present disclosure, the terms such as “first”, “second”, and “third” are used merely as labels and are not intended to impose ordinal requirements on the items to which these terms refer.

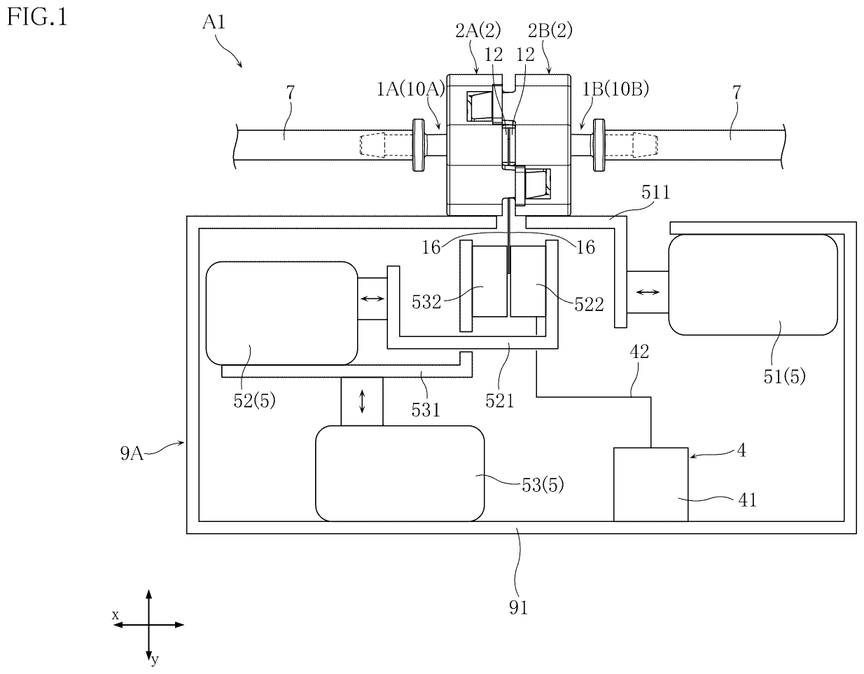

FIG. 1 shows a tube connection system according to an embodiment of the present disclosure. The tube connection system A1 of the present embodiment includes a first fluid-path member 1A, a second fluid-path member 1B, a holder 2, a heater 4, and an extractor. In the present embodiment, the tube connection system A1 is configured to connect a pair of tubes attached to the first fluid-path member 1A and the second fluid-path member 1B via the first fluid-path member 1A and the second fluid-path member 1B. The tube connection system A1 is not limited to any particular use, but may be used, for example, in the field of cell culture to connect a culture medium storage bag and a cell culture bag. In such a case, the culture medium storage bag is connected to an end of one of the pair of tubes 7, while the cell culture bag is connected to an end of the other tube.

The tube connection system A1 includes, for example, a connection apparatus 9A. The holder 2, the heater 4, and the extractor 5 are disposed at appropriate locations in the connection apparatus 9A. In the illustrated example, the connection apparatus 9A has a housing 91, and the heater 4 and the extractor 5 are disposed in the housing 91.

The holder 2 is disposed at the top of the housing 91. The holder 2 includes a first joint frame 2A and a second joint frame 2B, described later. The first joint frame 2A is removably fixed to the top of the housing 91. The second joint frame 2B is removably fixed to the top of a support member 511, described later. The first joint frame 2A and the second joint frame 2B are disposed next to each other in a first direction x (the horizontal direction in the figure). The first fluid-path member 1A and the second fluid-path member 1B are held by the holder 2 (the first joint frame 2A and the second joint frame 2B).

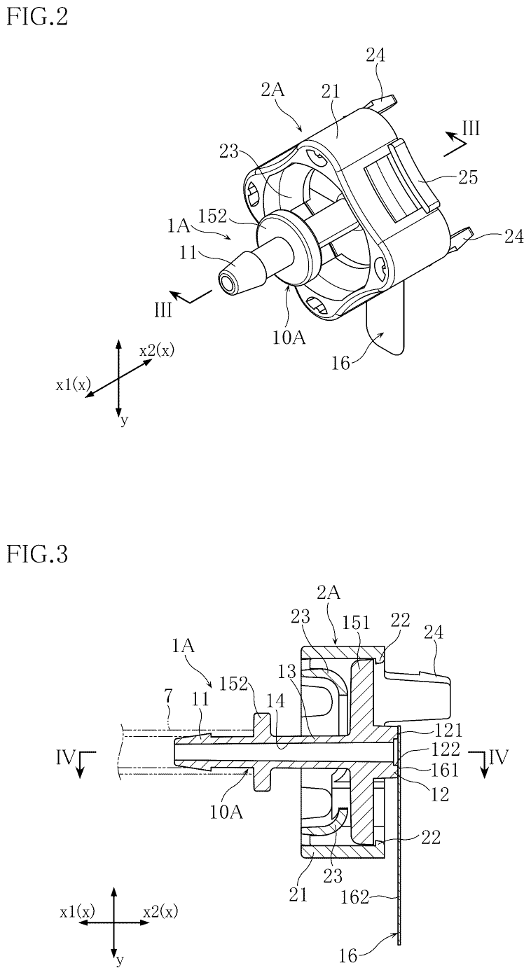

As shown in FIGS. 2 to 4, the first fluid-path member 1A includes a first tube connector 10A and a sealing element 16. The first tube connector 10A has a tube attachment portion 11, a fluid path end 12, a body 13, an internal channel 14, a flange portion 151, and a flange portion 152. As shown in FIGS. 5 to 7, the second fluid-path member 1B includes a second tube connector 10B and a sealing element 16. The second tube connector 10B has a tube attachment portion 11, a fluid path end 12, a body 13, an internal channel 14, a flange portion 151, and a flange portion 152. In the present embodiment, the first fluid-path member 1A and the second fluid-path 1B have the same structure. The member configuration of the first fluid-path member 1A (the second fluid-path member 1B) will be described mainly with reference to FIGS. 2 to 4.

The tube attachment portion 11 is disposed on one side in the first direction x (hereinafter referred to as the “first side x1 in the first direction” as appropriate). In the illustrated example, the tube attachment portion 11 is configured as a barb fitting, and the flexible tube 7 can be attached by press-fitting it over the tube attachment portion 11. The shape or the like of the tube attachment portion 11 is not limited to the illustrated example. For example, the tube attachment portion 11 may be configured such that the tube 7 is fitted into it. The tube 7 may be fixed with an adhesive in a state where it is fitted over or into the tube attachment portion 11.

The fluid path end 12 is spaced apart from the tube attachment portion 11 in the first direction x and located on the other side in the first direction x (hereinafter referred to as the “second side x2 in the first direction” as appropriate). The body 13 connects the tube attachment portion 11 and the fluid path end 12 in the first direction x. In the illustrated example, the body 13 is generally circular in cross section. The internal channel 14 is a through-hole penetrating from the tube attachment portion 11 to the fluid path end 12 in the first direction x. The flange portion 151 is a portion to engage with the first joint frame 2A (the second joint frame 2B). The flange portion 151 is provided near the fluid path end 12 and extends radially outward from the outer circumference of the body 13. The flange portion 152 is provided near the tube attachment portion 11 and extends radially outward from the outer circumference of the body 13. In the illustrated example, the flange portion 151 and the flange portion 152 are generally disk-shaped.

The fluid path end 12 has a first end surface 121 and a fluid path end recess 122. The first end surface 121 faces the second side x2 in the first direction and is an annular flat surface. The fluid path end recess 122 is recessed from the first end surface 121 toward the first side x1 in the first direction on the radially inner side of the first end surface 121. The diameter of the fluid path end recess 122 is larger than that of the internal channel 14, and the fluid path end recess 122 surrounds the internal channel 14 as viewed in the first direction x. Unlike the illustrated example, the fluid path end 12 may not have the fluid path end recess 122.

The fluid path end 12 is made of a material that can be thermally bonded by fusion bonding, for example. In the present embodiment, the tube attachment portion 11, the fluid path end 12, the body 13, and the flange portions 151 and 152 are integrally formed of a synthetic resin that can be fusion-bonded. Examples of the resin material that can be fusion-bonded include, without limitation, thermoplastic resins with a relatively low melting point, such as polyethylene and polyolefin elastomer (POE). Unlike the present embodiment, a portion that includes the first end surface 121 of the fluid path end 12 may be made of a material that can be fusion-bonded, while the remaining portion may be made of a different material (e.g., a resin material with a higher heat resistant temperature or a metal material). For example, a portion that includes the first end surface 121 of the fluid path end 12 may be made of elastomer that can be fusion-bonded, while the remaining portion may be made of polycarbonate, and these portions may be integrated by double-molding.

The sealing element 16 is a component that covers the fluid path end 12 and has a higher thermal conductivity than that of the fluid path end 12. The sealing element 16 includes a thin metal plate or a thin metal film. In the present embodiment, the sealing element 16 is a multilayer body that includes a thin metal film. The sealing element 16 may be composed of aluminum foil as a thin metal film, and a resin film as a heat-sealing layer laminated on part of the aluminum foil (the first portion 161, described later). The heat sealing layer of the sealing element 16 is fusion-bonded and held in close contact with the first end surface 121 of the fluid path end 12, covering the first end surface 121.

Unlike the present embodiment, the sealing element 16 may have a configuration, like an aluminum vapor deposition film, provided by applying a thin metal film to a resin film by coating, plating or the like. Alternatively, the sealing element 16 may be made of a thin metal plate or a thin metal film alone. In such a case, the sealing element 16 (a thin metal plate or a thin metal film alone) and the first end surface 121 of the fluid path end 12 are joined by fusion bonding. The thin metal plate is not limited to a particular material, and may be, for example, a thin copper plate or a thin aluminum plate. The sealing element 16 may not include a thin metal plate or a thin metal film. The sealing element 16 may be a thin plate made of a ceramic material with high thermal conductivity, such as alumina.

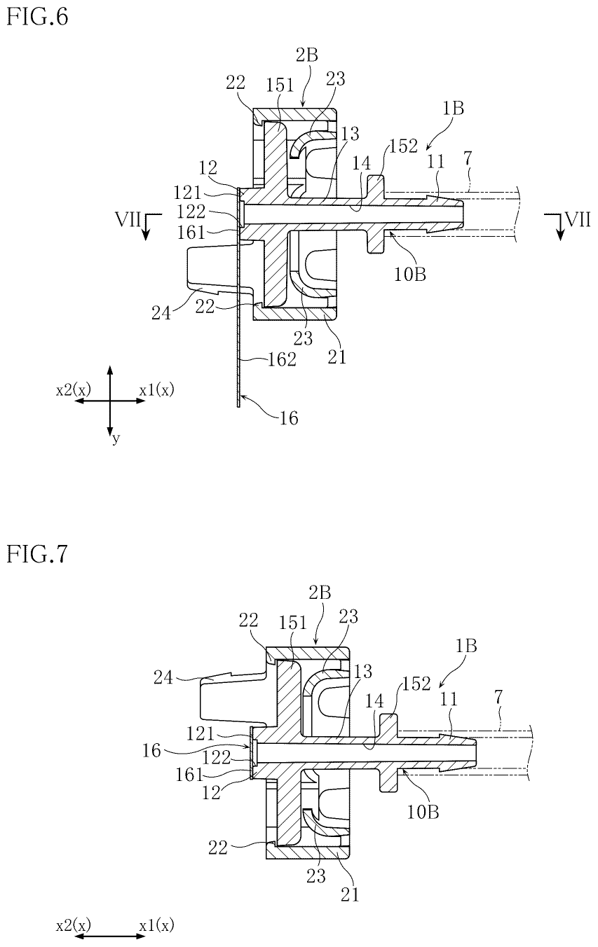

The sealing element 16 includes a first portion 161 and a second portion 162. The first portion 161 is a portion that overlaps with the fluid path end 12 as viewed in the first direction x. In the present embodiment, the first portion 161 is fusion-bonded to the first end surface 121 of the fluid path end 12 to cover the first end surface 121. The second portion 162 is connected to the first portion 161 and extends from the first portion 161 in a direction orthogonal to the first direction x (downward in FIGS. 3 and 6).

As shown in FIGS. 2 to 4, the first joint frame 2A includes a tubular body 21, a drop prevention claw 22, an elastic function portion 23, an engaging claw 24, and an engaged portion 25. As shown in FIGS. 5 to 7, the second joint frame 2B includes a tubular body 21, a drop prevention claw 22, an elastic function portion 23, an engaging claw 24, and an engaged portion 25. In the present embodiment, the first joint frame 2A and the second joint frame 2B have the same configuration. The first joint frame 2A holds the first fluid-path member 1A, and the second joint frame 2B holds the second fluid-path member 1B. As will be described later, the first joint frame 2A and the second joint frame 2B can be fitted together and integrated. The configuration of the first joint frame 2A (the second joint frame 2B) will be described mainly with reference to FIGS. 2 to 4.

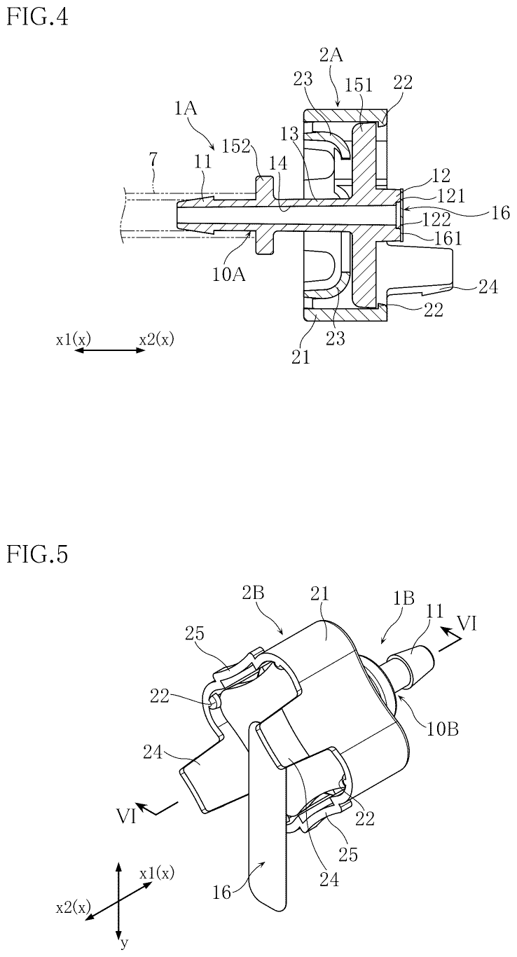

The tubular body 21 has the shape of a roughly square tube. The drop prevention claw 22 protrudes inward from the end of the tubular body 21 on the second side x2 in the first direction. In the illustrated example, the first joint frame 2A has a plurality of drop prevention claws 22 that are spaced apart from each other. The elastic function portion 23 extends inside the tubular body 21 from near the end of the tubular body 21 on the first side x1 in the first direction toward the second side x2 in the first direction. In the illustrated example, the first joint frame 2A has a plurality of elastic function portions 23 spaced apart from each other. Each elastic function portion 23 has a distal end curved toward the inner side of the first joint frame 2A. As will be described later, when the first joint frame 2A and the second joint frame 2B are fitted together and integrated, the elastic function portions 23 push the first tube connector 10A and the second tube connector 10B in a direction to bring them close to each other. As shown in FIGS. 3 and 4, before the first joint frame 2A and the second joint frame 2B are fitted together, the flange portion 151 is located between the drop prevention claw 22 and the elastic function portion 23 in the first direction x. This prevents the first tube connector 10A from dropping off the first joint frame 2A, and the first tube connector 10A (the second tube connector 10B) is temporarily held by the first joint frame 2A (the second joint frame 2B). The shape of the elastic function portion 23 is not limited to the illustrated example, and can be changed in various ways. Although the tubular body 21 is described as having the shape of a roughly square tube in the illustrated example, the shape of the tubular body 21 is not limited. For example, the tubular body 21 may have the shape of a roughly octagonal tube or a roughly cylindrical tube.

The engaging claw 24 extends outward from the end on the second side x2 in the first direction of the tubular body 21 toward the second side x2 in the first direction. The engaged portion 25 is provided at the end on the second side x2 in the first direction of the tubular body 21. The engaging claw 24 can be fitted into the engaged portion 25. In the illustrated example, the first joint frame 2A (the second joint frame 2B) has a pair of engaging claws 24 and a pair of engaged portions 25. The pair of engaging claws 24 are disposed at intervals of 180° in the circumferential direction of the tubular body 21. The pair of engaged portions 25 are disposed at intervals of 180° in the circumferential direction of the tubular body 21. The engaging claws 24 and the engaged portions 25 are alternately disposed in the circumferential direction of the tubular body 21 and disposed at equal intervals of 90° in the circumferential direction of the tubular body 21. As understood from FIGS. 2, 5, and 8, the first joint frame 2A and the second joint frame 2B can be fitted together and integrated by bringing the first joint frame 2A and the second joint frame 2B close together, with the engaging claws 24 (the engaged portions 25) of the first joint frame 2A facing the engaged portions 25 (the engaging claws 24) of the second joint frame 2B. The first joint frame 2A (the second joint frame 2B) having the above configuration is integrally formed from synthetic resin, for example. Examples of the synthetic resin material that forms the first joint frame 2A (the second joint frame 2B) include polycarbonate.

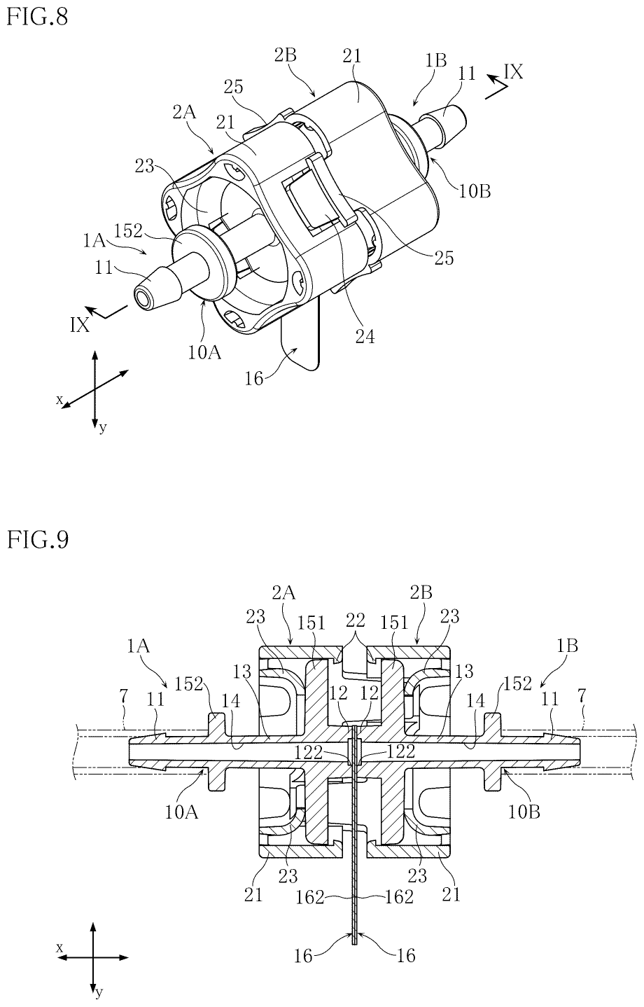

FIGS. 8 to 10 show the first joint frame 2A and the second joint frame 2B fitted together and integrated. As understood from these figures, the pair of engaged portions 25 (the pair of engaging claws 24) of the first joint frame 2A and the pair of engaging claws 24 (the pair of engaged portions 25) of the second joint frame 2B are individually fitted to each other, whereby the first joint frame 2A and the second joint frame 2B are integrated. In this state, the tube attachment portion 11 of each of the first tube connector 10A and the second tube connector 10B is located on the outer side in the first direction x. The sealing elements 16 of the first fluid-path member 1A and the second fluid-path member 1B are held in contact with each other. The elastic function portions 23 are pressed against the flange portions 151 of the first tube connector 10A and the second tube connector 10B, and the elastic restoring force of the elastic function portions 23 pushes the flange portions 151 of the first tube connector 10A and the second tube connector 10B from the outer side toward the inner side in the first direction x. As a result, the sealing elements 16 of the first fluid-path member 1A and the second fluid-path member 1B are pressed against each other. Thus, when the first joint frame 2A and the second joint frame 2B are fitted together and integrated, the first tube connector 10A and the second tube connector 10B can be held with the sealing elements 16 of the first fluid-path member 1A and the second fluid-path member 1B facing and being pressed against each other.

In the connection apparatus 9A shown in FIG. 1, the extractor 5 functions to pull out the sealing element 16 of each of the first fluid-path member 1A and the second fluid-path member 1B. In the illustrated example, the extractor 5 includes actuators 51, 52, and 53. The actuators 51 to 53 are not limited to a particular configuration, but are magnetic solenoids in the illustrated example.

A support member 511 is fixed to the rod of the actuator 51. The second joint frame 2B is removably fixed to the top of the support member 511. The actuator 51 moves the second joint frame 2B, which is fixed to the support member 511, along the first direction x. A support member 521 is fixed to the rod of the actuator 52. A seal clamping member 522 is fixed to the support member 521. The actuator 52 moves the seal clamping member 522, which is fixed to the support member 521, along the first direction x. A support member 531 is fixed to the rod of the actuator 53. The actuator 52 and a seal clamping member 532 are fixed to the support member 531. The seal clamping member 532 is disposed to face the seal clamping member 522 in the first direction x. The actuator 53 moves the actuator 52 and the seal clamping member 532 in the second direction y.

The seal clamping members 522 and 532 described above are the parts that hold the sealing elements 16 from both sides when pulling out the sealing elements 16. To increase the holding force for the sealing elements 16, the mutually opposing surfaces of the seal clamping members 522 and 532 may be formed with irregularities or the like to improve the anti-slip function.

The heater 4 is configured to heat the sealing element 16 of each of the first fluid-path member 1A and the second fluid-path member 1B. In the example shown in FIG. 1, the heater 4 includes a heat source 41 and a heat transfer section 42. The heat source 41 is not limited to a particular configuration, but may be a heating device (nichrome wire heating device or ceramic heating device) that uses a resistor element that generates heat when an electric current is applied, or may use friction heat from ultrasonic vibrations or electromagnetic induction heating as appropriate. The heat transfer section 42, which is in contact with both the heat source 41 and the seal clamping member 522, is made of a material with excellent thermal conductivity. The seal clamping member 522, with which the heat transfer section 42 is in contact, functions as a heat transfer element to the sealing elements 16. The seal clamping members 522 and 532, which clamp the sealing elements 16, may be made of a metal material with excellent thermal conductivity. As shown in FIG. 1, when the first joint frame 2A and the second joint frame 2B are fitted together and integrated, the sealing elements 16 of the first fluid-path member 1A and the second fluid-path member 1B are in contact with each other. The sealing elements 16 are heated by the heat transferred from the heat source 41 through the heat transfer section 42 and the seal clamping member 522.

Next, the process of connecting the first tube connector 10A and the second tube connector 10B using the tube connection system A1 will be described with reference to FIG. 1.

First, the first joint frame 2A is fixed to the top of the housing 91, and the second joint frame 2B is fixed to the top of the support member 511. At this time, the support member 511 is located further to the right than in the state shown in FIG. 1, and the first joint frame 2A and the second joint frame 2B are spaced apart from each other. Also, the support member 521, to which the seal clamping member 522 is fixed, is located further to the right than in the state shown in FIG. 1, and there is sufficient space between the seal clamping member 522 and the seal clamping member 532.

Next, the actuator 51 is operated to move the support member 511 and the second joint frame 2B fixed to the support member 511 toward the first joint frame 2A side (the left side in FIG. 1). As a result, the first joint frame 2A and the second joint frame 2B are fitted together and fixed in an integrated state as shown in FIG. 1. At this time, the sealing element 16 of the first fluid-path member 1A and the sealing element 16 of the second fluid-path member 1B are pressed against each other while facing each other.

Next, the actuator 52 is operated to move the seal clamping member 522, which is fixed to the support member 521, toward the seal clamping member 532 side (the left side in FIG. 1). As a result, the sealing elements 16 of the first fluid-path member 1A and the second fluid-path member 1B are clamped by the seal clamping members 522 and 532 as shown in FIG. 1. In this state, the heater 4 heats the sealing element 16 of each of the first fluid-path member 1A and the second fluid-path member 1B via the seal clamping member 522. As a result, the sealing elements 16 and the first end surfaces 121 of the fluid path ends 12 covered with the sealing elements 16 are heated, and the first end surfaces 121 are heated to a state where they can be fusion-bonded. In the illustrated example, the sealing elements 16 of the first fluid-path member 1A and the second fluid-path member 1B, which are pressed against each other, are heated by the single seal clamping member 522 (the heat transfer element) that is in contact with the sealing element 16 of the second fluid-path member 1B. However, the present disclosure is not limited to such a configuration. For example, the heat transfer section 42 may be connected to the seal clamping member 532 as well, and the sealing elements 16 may be heated by the seal clamping members 522 and 532 that are individually in contact with the sealing elements 16 of the first fluid-path member 1A and the second fluid-path member 1B.

Next, the actuator 53 is operated to move the seal clamping member 532 fixed to the support member 531 and the actuator 52 fixed to the support member 531 in the second direction y (downward in FIG. 1). At this time, the seal clamping member 522 is supported by the actuator 52 via the support member 521, the seal clamping members 522 and 532 move simultaneously in the second direction y. Thus, the pair of sealing elements 16 clamped by the seal clamping members 522 and 532 are pulled out in the second direction y (downward in FIG. 1). At the same time as the pair of sealing elements 16 are pulled out, the second tube connector 10B is pushed toward the first tube connector 10A by the pushing operation of the actuator 51. Thus, the fluid path ends 12 (the first end surfaces 121) of the first tube connector 10A and the second tube connector 10B are pressed against each other. If the actuator 51 does not have the function of continuously pushing the second tube connector 10B toward the first tube connector 10A, the first tube connector 10A and the second tube connector 10B are pressed against each other inward in the first direction x by the elastic restoring force of the elastic function portions 23 of the first joint frame 2A and the second joint frame 2B. Thus, the fluid path ends 12 (the first end surfaces 121) of the first tube connector 10A and the second tube connector 10B are pressed against each other. The first end surfaces 121 of the fluid path ends 12 have been heated to a state where they can be fusion-bonded. Thus, the first end surfaces 121 of the fluid path ends 12, which are pressed against each other, are joined together by fusion bonding. In this way, the fluid path ends 12 of the first tube connector 10A and the second tube connector 10B are connected. Thereafter, the first joint frame 2A and the second joint frame 2B are removed from the housing 91 and the support member 511.

FIG. 11 shows the first tube connector 10A and the second tube connector 10B connected together. As shown in FIG. 11, when the fluid path ends 12 of the first tube connector 10A and the second tube connector 10B are connected, the internal channel 14 of the first tube connector 10A and the internal channel 14 of the second tube connector 10B communicate with each other. The pair of left and right tubes 7 attached to the tube attachment portions 11 of the first tube connector 10A and the second tube connector 10B are connected via the first tube connector 10A and second tube connector 10B. FIG. 11, the first end surface 121 of the fluid path end 12 of each of the first tube connector 10A and the second tube connector 10B is shown clearly for the convenience of drawing. Conceivably, the actual thickness of each fluid path end 12 becomes smaller by the first end surfaces 121 being integrally joined together by fusion bonding. The same applies to variations described later.

Next, the effects of the present embodiment will be described.

In the tube connection system A1 of the present embodiment, each of the first fluid-path member 1A and the second fluid-path member 1B has the internal channel 14 penetrating in the first direction x, the fluid path end 12 located at an end of the internal channel 14, and the sealing element 16 covering the fluid path end 12. The fluid path end 12 can be fusion-bonded, and the sealing element 16 has a higher thermal conductivity than that of the fluid path end 12. The sealing elements 16 of the first fluid-path member 1A and the second fluid-path member 1B are held pressed against each other by the holder 2 (the first joint frame 2A and the second joint frame 2B). Also, the sealing elements 16 of the first fluid-path member 1A and the second fluid-path member 1B can be heated by the heater 4, and can be pulled out in a direction (the second direction y) orthogonal to the first direction x by the extractor 5. With such a configuration, by operating the heater 4 and the extractor 5 as appropriate, the heated fluid path ends 12 of the first fluid-path member 1A (the first tube connector 10A) and the second fluid-path member 1B (the second tube connector 10B) can be joined together by fusion bonding, as described with reference to FIG. 1. The fluid path ends 12 are joined together at the same time as the sealing elements 16 are pulled out. Therefore, the first tube connector 10A and the second tube connector 10B are aseptically connected to each other at the fluid path ends 12 without bacteria or foreign matter mixing in from the outside air.

In the present embodiment, the first fluid-path member 1A includes the first tube connector 10A, and the second fluid-path member 1B includes the second tube connector 10B. Each of the first tube connector 10A and the second tube connector 10B has the tube attachment portion 11 disposed on the first side x1 in the first direction, the fluid path end 12 disposed on the second side x2 in the first direction, and the internal channel 14 penetrating from the tube attachment portion 11 to the fluid path end 12 in the first direction x. Such a configuration allows the first tube connector 10A and the second tube connector 10B to be aseptically connected to each other at the fluid path ends 12 without bacteria or foreign matter mixing in from the outside air, as described above. Therefore, the pair of tubes 7 attached to the tube attachment portions 11 of the first tube connector 10A and the second tube connector 10B can be aseptically connected via the first tube connector 10A and the second tube connector 10B.

The first tube connector 10A and the second tube connector 10B of the tube connection system A1 can select different materials for the pair of tubes 7 to be attached to the tube attachment portions 11 as appropriate. According to the present embodiment, therefore, a pair of tubes 7 made of different materials can be properly joined together via the first tube connector 10A and the second tube connector 10B.

The holder 2 includes the first joint frame 2A holding the first tube connector 10A and the second joint frame 2B holding the second tube connector 10B. The first joint frame 2A and the second joint frame 2B, when fitted together and integrated, hold the first tube connector 10A and the second tube connector 10B with the sealing elements 16 of the first fluid-path member 1A and the second fluid-path member 1B facing and being pressed against each other. With such a configuration, the relative positional relationship between the first tube connector 10A held in the first joint frame 2A and the second tube connector 10B held in the second joint frame 2B can be properly fixed.

In the present embodiment, the first joint frame 2A and the second joint frame 2B have the elastic function portions 23. The elastic function portions 23 cause the sealing elements 16 of the first joint frame 2A and the second joint frame 2B to be pressed against each other. With such a configuration, in connecting the first tube connector 10A and the second tube connector 10B, at the same time as the pair of sealing elements 16 are pulled out by the extractor 5, the elastic restoring force of the elastic function portions 23 pushes the first tube connector 10A and the second tube connector 10B toward each other inward in the first direction x. The fluid path ends 12 (the first end surfaces 121) of the first tube connector 10A and the second tube connector 10B are pressed against each other. Thus, the first end surfaces 121 of the fluid path ends 12, which are pressed against each other, can be properly joined together by fusion bonding.

In the present embodiment, each fluid path end 12 has the first end surface 121 and the fluid path end recess 122. The fluid path end recess 122 is recessed from the first end surface 121 toward the first side x1 in the first direction on the radially inner side of the first end surface 121. The fluid path end recess 122 surrounds the internal channel 14 as viewed in the first direction x. With the configuration having the fluid path end recess 122, if film-like burrs are generated at the first end surface 121 due to molten resin when the sealing element 16 covering the first end surface 121 of fluid path end 12 is pulled out, it is possible to prevent the burrs from blocking the internal channel 14.

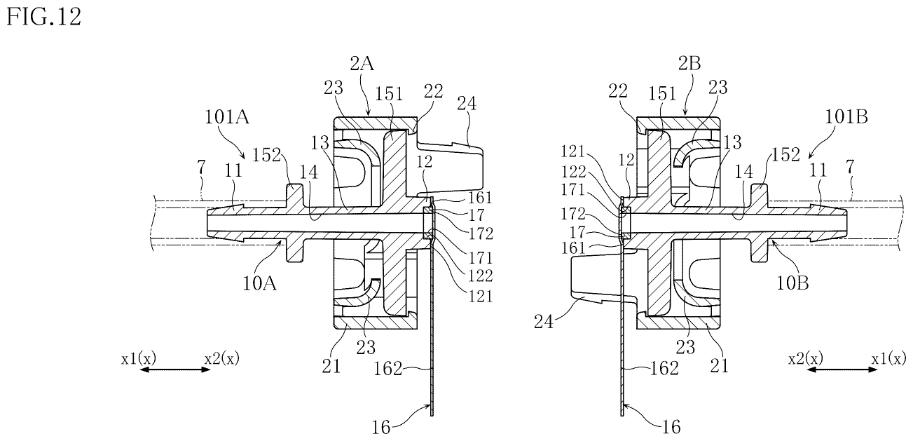

FIG. 12 shows a first variation of the first fluid-path member 1A and the second fluid-path member 1B shown in the above embodiment. FIG. 12 is a sectional view of the first fluid-path member 101A and the second fluid-path member 101B according to the first variation and corresponds to the sectional views shown in FIGS. 3 and 6. In FIG. 12 and the subsequent figures, the elements that are identical or similar to those of the first fluid-path member 1A (the second fluid-path member 1B) and the first joint frame 2A (the second joint frame 2B) are denoted by the same reference signs, and the descriptions thereof are omitted as appropriate. The structures of various parts of the variations shown in FIG. 12 and the subsequent figures may be selectively used in any appropriate combination as long as it is technically compatible.

As shown in FIG. 12, in the first fluid-path member 101A and the second fluid-path member 101B of the present variation, the fluid path end 12 of each of the first tube connector 10A and the second tube connector 10B has the first end surface 121 and the fluid path end recess 122, as with the first tube connector 10A and the second tube connector 10B of the above embodiment. In the present variation, each of the first tube connector 10A and the second tube connector 10B has an annular elastic element 17 attached to the fluid path end recess 122. The annular elastic element 17 has a first through-hole 171 and a second end surface 172. The first through-hole 171 communicates with the internal channel 14. The second end surface 172 faces the second side x2 in the first direction, and is located on the second side x2 in the first direction from the first end surface 121 of the fluid path end 12 in the natural state. The sealing element 16 (the first portion 161) covers the first end surface 121 of the fluid path end 12 and the second end surface 172 of the annular elastic element 17. The annular elastic element 17 has a higher heat resistant temperature than that of the fluid path end 12 (the first end surface 121). The annular elastic element 17 is made of a soft material that is elastically deformable. The constituent material of the annular elastic element 17 is not limited, and may be silicone rubber, for example. The first tube connector 10A and the second tube connector 10B of the present variation can be connected to each other in the same way as the connection procedure for the first tube connector 10A and the second tube connector 10B of the above embodiment described with reference to FIG. 1.

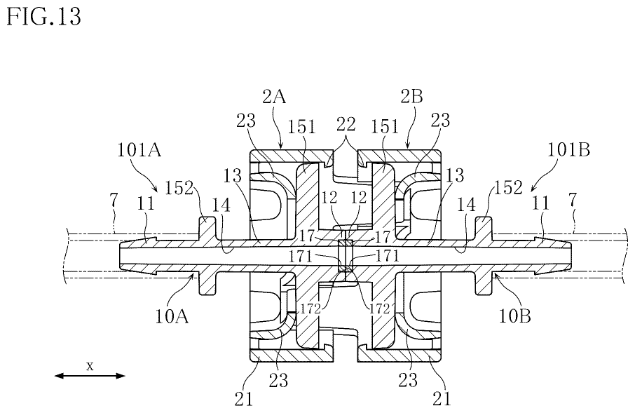

FIG. 13 shows the first tube connector 10A and the second tube connector 10B of the present variation connected together. As shown in FIG. 13, when the fluid path ends 12 of the first tube connector 10A and the second tube connector 10B are connected, the internal channel 14 of the first tube connector 10A and the internal channel 14 of the second tube connector 10B communicate with each other through the first through-holes 171. The pair of tubes 7 attached to the tube attachment portions 11 of the first tube connector 10A and the second tube connector 10B are connected via the first tube connector 10A and the second tube connector 10B. The first tube connector 10A and the second tube connector 10B are aseptically connected to each other at the fluid path ends 12 without bacteria or foreign matter mixing in from the outside air. Therefore, the pair of tubes 7 attached to the tube attachment portions 11 of the first tube connector 10A and the second tube connector 10B can be aseptically connected via the first tube connector 10A and the second tube connector 10B.

In the present variation, the annular elastic element 17 is attached to the fluid path end recess 122, and in the natural state, the second end surface 172 of the annular elastic element 17 protrudes toward the second side x2 in the first direction relative to the first end surface 121 of the fluid path end 12. With such a configuration, in the state where the first tube connector 10A and the second tube connector 10B are connected as shown in FIG. 13, the fluid path ends 12 (the first end surfaces 121) of the first tube connector 10A and the second tube connector 10B are joined together by fusion bonding. The annular elastic elements 17 of the first tube connector 10A and the second tube connector 10B are compressed against each other, and the second end surfaces 172 are pressed against each other. According to the present variation, the annular elastic elements 17 improve the sealing performance at the connection point of the first tube connector 10A and the second tube connector 10B, and leakage of fluids or the like at the connection point is properly prevented. Additionally, the present variation achieves the same effect as the above-described embodiment.

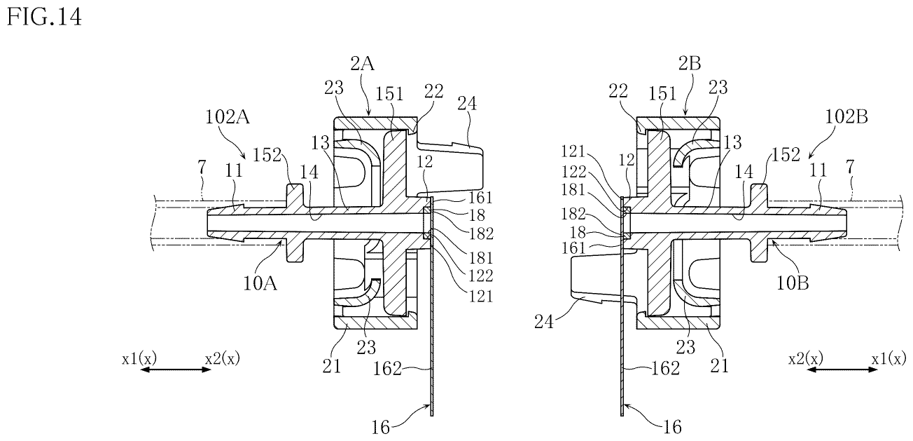

FIG. 14 shows a second variation of the first fluid-path member 1A and the second fluid-path member 1B shown in the above embodiment. FIG. 14 is a sectional view of the first fluid-path member 102A and the second fluid-path member 102B according to the second variation and corresponds to the sectional views shown in FIGS. 3 and 6.

As shown in FIG. 14, in the first fluid-path member 102A and the second fluid-path member 102B of the present variation, the fluid path end 12 of each of the first tube connector 10A and the second tube connector 10B has the first end surface 121 and the fluid path end recess 122, as with the first tube connector 10A and the second tube connector 10B of the above embodiment. In the present variation, each of the first tube connector 10A and the second tube connector 10B has an annular element 18 attached to the fluid path end recess 122. The annular element 18 has a second through-hole 181 and a third end surface 182. The second through-hole 181 communicates with the internal channel 14. The third end surface 182 faces the second side x2 in the first direction. The sealing element 16 (the first portion 161) covers the first end surface 121 of the fluid path end 12 and the third end surface 182 of the annular element 18. The annular element 18 only needs to have a higher heat resistant temperature than that of the fluid path end 12 (the first end surface 121), and the constituent material of the annular element 18 is not particularly limited. For example, when the constituent material of the fluid path end 12 (the first end surface 121) is polyethylene, polypropylene, which has a higher heat resistant temperature (higher melting point) than that of polyethylene, may be used as the constituent material of the annular element 18. The first tube connector 10A and the second tube connector 10B of the present variation can be connected to each other in the same way as the connection procedure for the first tube connector 10A and the second tube connector 10B of the above embodiment described with reference to FIG. 1.

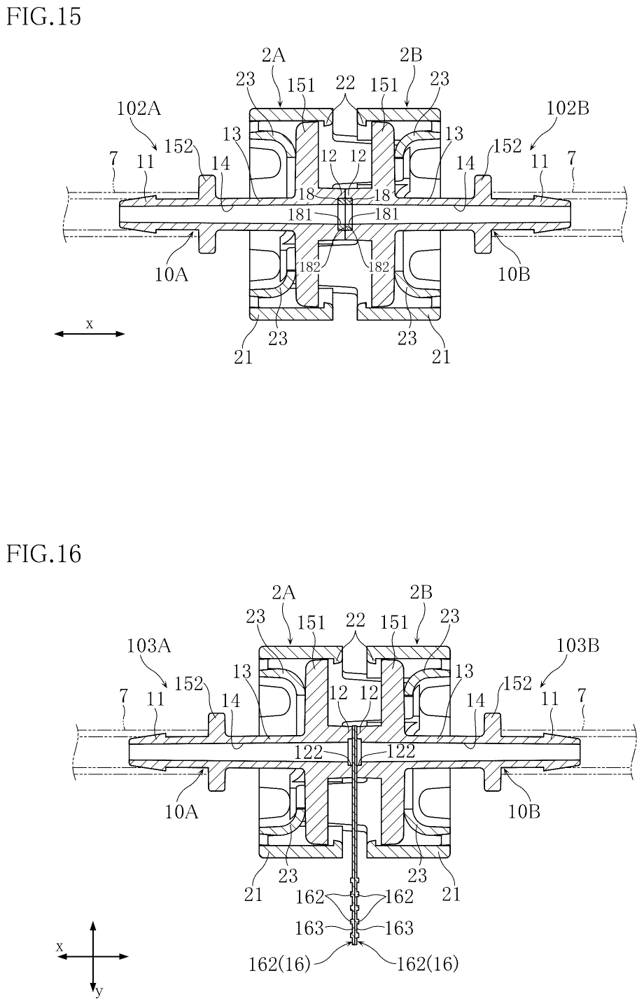

FIG. 15 shows the first tube connector 10A and the second tube connector 10B of the present variation connected together. As shown in FIG. 15, when the fluid path ends 12 of the first tube connector 10A and the second tube connector 10B are connected, the internal channel 14 of the first tube connector 10A and the internal channel 14 of the second tube connector 10B communicate with each other through the second through-holes 181. The pair of tubes 7 attached to the tube attachment portions 11 of the first tube connector 10A and the second tube connector 10B are connected via the first tube connector 10A and the second tube connector 10B. The first tube connector 10A and the second tube connector 10B are aseptically connected to each other at the fluid path ends 12 without bacteria or foreign matter mixing in from the outside air. Therefore, the pair of tubes 7 attached to the tube attachment portions 11 of the first tube connector 10A and the second tube connector 10B can be aseptically connected via the first tube connector 10A and the second tube connector 10B.

In the present variation, the annular element 18 is attached to each fluid path end 12, and the first end surface 121 of the fluid path end 12 and the third end surface 182 of the annular element 18 are covered with the sealing element 16 (the first portion 161). With such a configuration, if burrs are generated at the first end surface 121 due to molten resin when the sealing element 16 covering the first end surface 121 of fluid path end 12 is pulled out, the third end surface 182 (the annular element 18) located radially inward of the first end surface 121 hinders the burrs from extending in a film-like manner. Thus, it is possible to prevent the burrs from blocking the second through-hole 181 communicating with the internal channel 14. Additionally, the present variation achieves the same effect as the above-described embodiment.

FIG. 16 shows a third variation of the first fluid-path member 1A and the second fluid-path member 1B shown in the above embodiment. FIG. 16 is a sectional view of the first fluid-path member 103A and the second fluid-path member 103B according to the third variation. FIG. 16 shows the state where the first joint frame 2A and the second joint frame 2B are fitted together and integrated, and is a sectional view corresponding to FIG. 9.

As shown in FIG. 16, in the first fluid-path member 103A and the second fluid-path member 103B of the present variation, the second portion 162 of the sealing element 16 of each of the first tube connector 10A and the second tube connector 10B has a first surface 163 facing the first side x1 in the first direction, and the first surface has a plurality of protrusions 164. The protrusions 164 are spaced apart from each other in the second direction y. Each of the protrusions 164 is a strip-shaped protrusion extending in a direction orthogonal to both the first direction x and the second direction y. Such a configuration increases the gripping force in pulling out the pair of sealing elements 16 by the seal clamping members 522 and 532, and the protrusions 164 on the first surface 163 provide an anti-slip effect in pulling out the sealing elements 16. The protrusions 164 on the first surface 163 is an example of the “anti-slip structure” of the present disclosure. The specific configuration of the anti-slipping structure is not limited to the protrusions 164 described above. The anti-slip structure can take various forms, such as roughening of the first surface 163 by graining, embossing, blasting, etching or the like, or printing, painting, coating, adhesion of a different material, fusing of a different material, or insert molding of a different material.

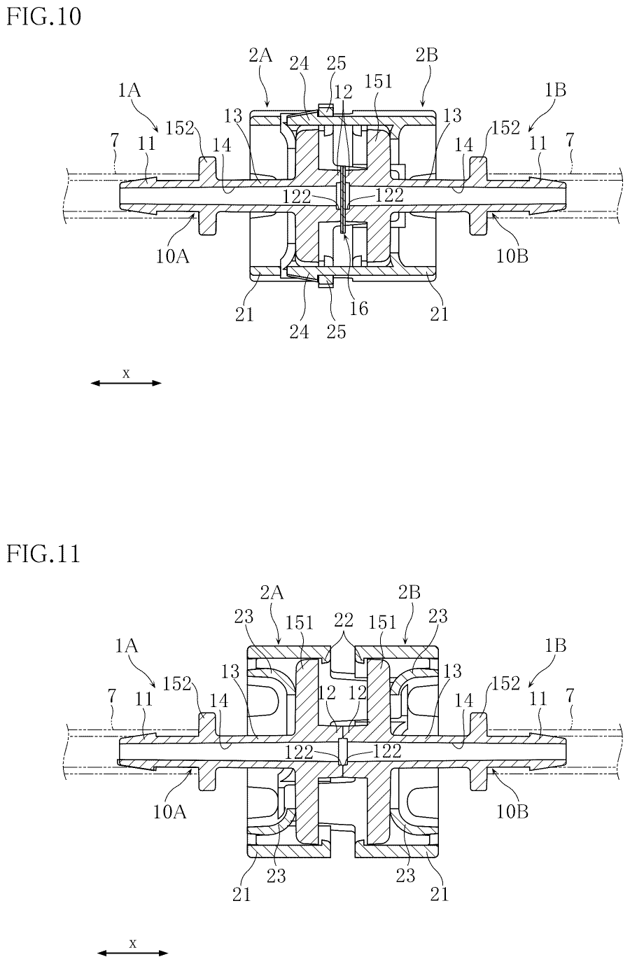

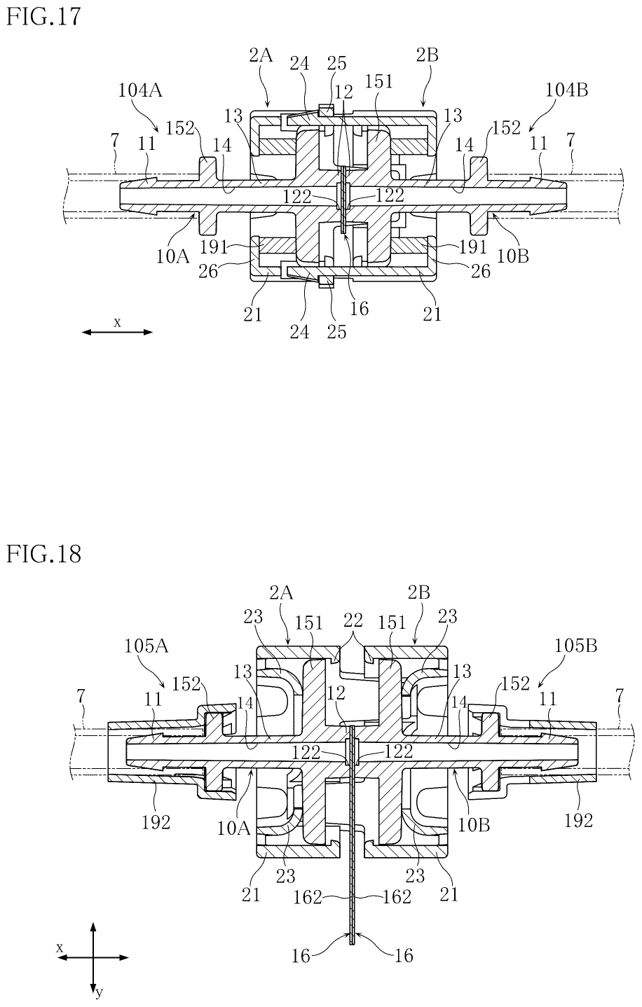

FIG. 17 shows a fourth variation of the first fluid-path member 1A and the second fluid-path member 1B shown in the above embodiment. FIG. 17 is a sectional view of the first fluid-path member 104A and the second fluid-path member 104B according to the fourth variation. FIG. 17 shows the first joint frame 2A and the second joint frame 2B fitted together and integrated, and is a sectional view corresponding to FIG. 10.

As shown in FIG. 17, in the first fluid-path member 104A and the second fluid-path member 104B of the present variation, each of the first tube connector 10A and the second tube connector 10B has the elastic function portion 191. The elastic function portion 191 is integrally attached to the flange portion 151 of each of the first tube connector 10A and the second tube connector 10B. In the illustrated example, the elastic function portion 191 is cylindrical. Each of the first joint frame 2A and the second joint frame 2B has a flange portion 26 extending radially inward from the end on the first side x1 in the first direction of the tubular body 21. The constituent material of the elastic function portion 191 is not limited, and may be silicone rubber, for example.

When the first joint frame 2A and the second joint frame 2B are fitted together and integrated, the elastic function portions 191 are pressed against the flange portions 26 of the first joint frame 2A and the second joint frame 2B. The elastic restoring force of the elastic function portions 191 pushes the flange portions 151 of the first tube connector 10A and the second tube connector 10B from the outer side toward the inner side in the first direction x. As a result, the sealing elements 16 of the first fluid-path member 104A and the second fluid-path member 104B are pressed against each With such a configuration, in connecting the first other. tube connector 10A and the second tube connector 10B, at the same time as the pair of sealing elements 16 are pulled out by the extractor 5, the elastic restoring force of the elastic function portions 191 pushes the first tube connector 10A and the second tube connector 10B toward each other inward in the first direction x. The fluid path ends 12 (the first end surfaces 121) of the first tube connector 10A and the second tube connector 10B are pressed against each other. Thus, the first end surfaces 121 of the fluid path ends 12, which are pressed against each other, can be properly joined together by fusion bonding. The configuration of the elastic function portions 191 is not limited to the illustrated example. The elastic function portions do not necessarily need to be provided integrally on the first tube connector 10A (the second tube connector 10B).

FIG. 18 shows a fifth variation of the first fluid-path member 1A and the second fluid-path member 1B shown in the above embodiment. FIG. 18 is a sectional view of the first fluid-path member 105A and the second fluid-path member 105B according to the fifth variation. FIG. 18 shows the first joint frame 2A and the second joint frame 2B fitted together and integrated, and is a sectional view corresponding to FIG. 9.

As shown in FIG. 18, each of the first fluid-path member 105A and the second fluid-path member 105B of the present variation has a tube stopper 192. The tube stopper 192 is attached to the first tube connector 10A (the second tube connector 10B) in such a manner that the tube 7 fitted over the tube attachment portion 11 is interposed between the tube stopper and the tube attachment portion. Though the detailed illustration is omitted, the tube stopper 192 has an engaging claw, and the engaging claw engages the flange portion 152. With such a configuration, the pair of tubes 7 attached to the tube attachment portions 11 is prevented from falling off the tube attachment portions 11 if unexpected external force or internal pressure is applied. Also, in some applications, the tube 7 and the tube attachment portion 11 may be made of materials that are difficult to be bonded together by an adhesive, such as where the tube 7 is made of soft polyvinyl chloride while the tube attachment portion 11 is made of polyethylene. Even in such a case, the tube 7 and the tube stopper 192 can be firmly bonded together when the tube stopper 192 is made of polycarbonate.

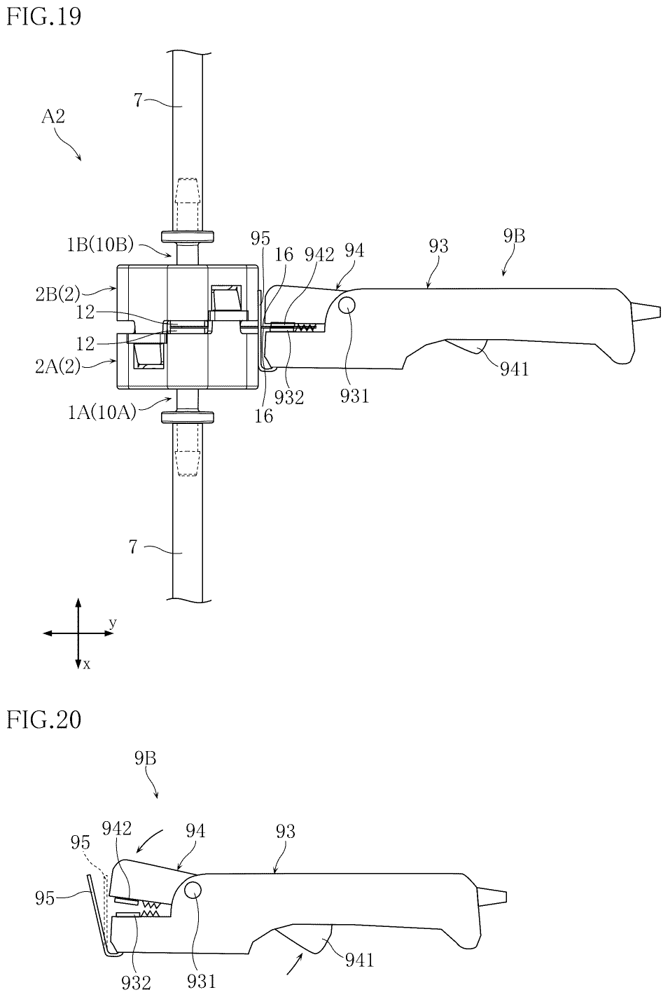

FIG. 19 shows a tube connection system according to another embodiment of the present disclosure. The tube connection system A2 of the present embodiment incudes a first fluid-path member 1A, a second fluid-path member 1B, a holder 2, a heater, and an extractor.

The tube connection system A2 includes, for example, a connection apparatus 9B. As shown in FIGS. 19 and 20, the connection apparatus 9B includes a handle 93, a movable element 94, and a thrust mechanism 95. The handle 93 is a part to be gripped by the user's hand, and has a seal clamping member 932 on its distal side. The movable element 94 is supported such that it can pivot around a pivot axis 931 relative to the handle 93, and has an operation lever 941 and a seal clamping member 942. The operation lever 941 is disposed on the proximal side of the movable element 94, and the seal clamping member 942 is located opposite from the operation lever 941 (on the distal side of the movable element 94) across the pivot axis 931. As shown in FIG. 20, when the user grips the operation lever 941 in the direction indicated by the arrow, the seal clamping member 942 disposed on the distal side of the movable element 94 comes into contact with the seal clamping member 932. As shown in FIG. 19, by placing a pair of sealing elements 16 between the seal clamping members 932 and 942 and gripping the operation lever 941, the sealing elements 16 can be clamped by the connection apparatus 9B (the handle 93 and the movable element 94). Though the detailed illustration is omitted, a biasing force by a spring or the like is applied to the connection apparatus 9B such that the operation lever 941 returns to the position shown in FIG. 20 when it is not gripped. A stopper mechanism may be provided to hold the operation lever 941 at the gripped position shown in FIG. 19. The stopper mechanism may be, for example, a ratchet structure.

The thrust mechanism 95 is configured to apply a pushing-off force to at least one of the first joint frame 2A and the second joint frame 2B when a pair of sealing elements 16 are clamped by the seal clamping members 932 and 942. The thrust mechanism 95 may be appropriately bent, extending from the bottom of the handle 93 on the distal side, and may be formed by a metal component having spring elasticity. The thrust mechanism 95 in the natural state is at the position shown by the solid lines in FIG. 20. When the thrust mechanism 95 is pushed to the right side in the figure to the position shown by the dotted lines in FIG. 20, the elastic restoring force in the direction (to the left in the figure) of returning to the natural state indicated by the solid lines is generated. The configuration of the thrust mechanism 95 is not limited to the illustrated example. Many variations are possible for the structure to push off the first joint frame 2A or the second joint frame 2B using a component with spring elasticity. For example, the thrust mechanism 95 may be provided using a compression coil spring.

Though the detailed illustration is omitted, in the connection apparatus 9B, the heater is disposed inside the handle 93. The heater includes, for example, a heat source and a heat transfer section, and the heat transfer section is in contact with both the heat source and the seal clamping member 932. The heat transfer section is made of a material with excellent thermal conductivity. The seal clamping member 932, with which the heat transfer section is in contact, functions as a heat transfer element to the sealing elements 16. The seal clamping members 932 and 942, which clamp the sealing elements 16, may be made of a metal material with excellent thermal conductivity. In the illustrated example, each of the opposing portions of the seal clamping members 932 and 942 includes a flat portion and an irregular portion, but the shape of the opposing portions of the seal clamping members 932 and 942 is not limited to this.

Next, the process of connecting the first tube connector 10A and the second tube connector 10B using the tube connection system A2 will be described.

First, the first fluid-path member 1A and the second fluid-path member 1B are held by the holder 2 (the first joint frame 2A and the second joint frame 2B), and the first joint frame 2A and the second joint frame 2B are fitted together and integrated as shown in FIGS. 8 to 10. Next, as shown in FIG. 19, while the distal side (the left side in the figure) of the connection apparatus 9B is pressed against the first joint frame 2A and the second joint frame 2B, the operation lever 941 is gripped, so that the pair of sealing elements 16 are clamped by the seal clamping members 932 and 942. At this time, the thrust mechanism 95 exerts elastic restoring force in the direction (to the left in FIG. 19) of returning to the natural state and pushes the first joint frame 2A and the second joint frame 2B to the left side in the figure.

In this state, the heater heats the sealing element 16 of each of the first fluid-path member 1A and the second fluid-path member 1B via the seal clamping member 932. As a result, the sealing elements 16 and the first end surfaces 121 of the fluid path ends 12 covered with the sealing elements 16 are heated, and the first end surfaces 121 are heated to a state where they can be fusion-bonded. Then, the fluid path ends 12 and the heat seal portions of the sealing elements 16 are melted, and the elastic restoring force of the thrust mechanism 95 pushes off the first joint frame 2A and the second joint frame 2B to the left side in the figure. Thus, the pair of sealing elements 16 clamped by the seal clamping members 932 and 942 are pulled out in the second direction y (to the right side in FIG. 19).

At the same time as the pair of sealing elements 16 are pulled out, the elastic restoring force of the elastic function portions 23 of the first joint frame 2A and the second joint frame 2B pushes the first tube connector 10A and the second tube connector 10B toward each other inward in the first direction x. The fluid path ends 12 (the first end surfaces 121) of the first tube connector 10A and the second tube connector 10B are pressed against each other. The first end surfaces 121 of the fluid path ends 12 have been heated to a state where they can be fusion-bonded. Thus, the first end surfaces 121 of the fluid path ends 12, which are pressed against each other, are joined together by fusion bonding. According to the present embodiment again, the first tube connector 10A and the second tube connector 10B are aseptically connected to each other at the fluid path ends 12 without bacteria or foreign matter mixing in from the outside air.

As described above, the tube connection system A2 includes the handle 93 and the movable element 94, and is capable of pulling out the sealing elements 16 by a relatively simple structure. The handle 93, the movable element 94, and the thrust mechanism 95 correspond to an example of the “extractor” of the present disclosure.

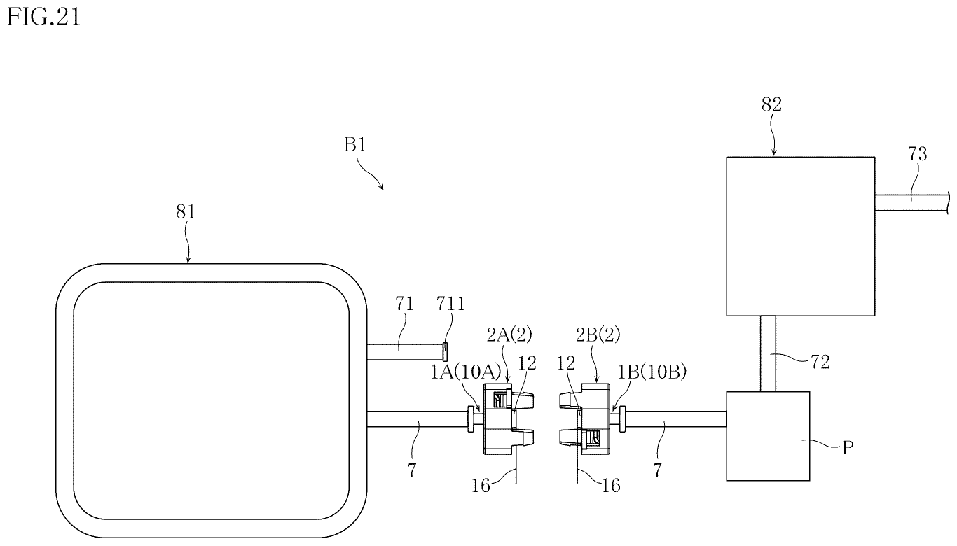

FIG. 21 is a schematic diagram showing an example of a tube connector set that constitutes the tube connection system according to the present disclosure. The tube connector set B1 shown in FIG. 21 includes a first fluid-path member 1A and a second fluid-path member 1B. In the illustrated example, the tube connector set B1 includes a pair of tubes 7, a culture medium storage bag 81, and a cell culture bag 82, in addition to the first fluid-path member 1A and the second fluid-path member 1B

The culture medium storage bag 81 is connected to an end of one of the tubes 7 that is attached to the first fluid-path member 1A (the first tube connector 10A). The culture medium storage bag 81 contains culture medium (liquid culture). The culture medium storage bag 81 is provided with a tube 71. The culture medium has been injected into the culture medium storage bag 81 through the tube 71 in an aseptic environment. The end of the tube 71 has a seal portion 711 formed by sealing the end by fusion bonding after the injection of the culture medium. A pump P is connected to an end of the other tube 7 attached to the second fluid-path member 1B (the second tube connector 10B). The cell culture bag 82 is connected to the pump P via the tube 72. The tube 72 is the inflow path for the culture medium. A tube 73 is connected to the cell culture bag 82. The tube 73 is a discharge path for the culture medium in the cell culture bag 82. A waste liquid bag, not shown, may be connected to an end of the tube 73.

The first tube connector 10A and the second tube connector 10B, which constitute the tube connector set B1, can be aseptically connected to each other in the same way as the connection procedure for the first tube connector 10A and the second tube connector 10B of the above embodiment described with reference to FIG. 1. According to the tube connector set B1, after the first tube connector 10A and the second tube connector 10B are aseptically connected, cell culture can be performed in the cell culture bag 82 by supplying culture medium from the culture medium storage bag 81 to the cell culture bag 82 via the first tube connector 10A, the second tube connector 10B, and the pump P.

Although a specific example of the configuration of the tube connector set B1 is shown in FIG. 21, the configuration of the tube connector set of the present disclosure is not limited to this. It is possible to connect various bags or containers to the ends of the pair of tubes 7 attached to the first fluid-path member 1A (the first tube connector 10A) and the second fluid-path member 1B (the second tube connector 10B).

Although specific embodiments of the present invention have been described above, the present invention is not limited to these, and various modifications are possible without departing from the spirit of the invention. Various modifications in design may be made freely in the specific structure of each part of the tube connection system according to the present invention.

Although the first fluid-path member 1A includes the first tube connector 10A while the second fluid-path member 1B includes the second tube connector 10B in the above embodiment, the configuration of the first fluid-path member and the second fluid-path member of the present disclosure is not limited to this. For example, each of the first fluid-path member and the second fluid-path member may include a tube and a sealing element. In such a case, an end of the tube corresponds to the fluid path end, and the fluid path end is covered with the sealing element. In both cases where the first fluid-path member (the second fluid-path member) includes the first tube connector (the second tube connector) and where the first fluid-path member (the second fluid-path member) includes a tube, various modifications to the holder are possible as long as the holder can hold the first fluid-path member and the second fluid-path member with their sealing elements facing and being pressed against each other.

### REFERENCE NUMERALS

A1, A2: Tube connection system B1: Tube connector set
1A, 101A, 102A, 103A, 104A, 105A: First fluid-path member
1B, 101B, 102B, 103B, 104B, 105B: Second fluid-path member
10A: First tube connector 10B: Second tube connector
11: Tube attachment portion 12: Fluid path end
121: First end surface 122: Fluid path end recess
13: Body 14: Internal channel 151, 152: Flange portion
16: Sealing element 161: First portion 162: Second portion
163: First surface 164: Protrusion (Anti-slip structure)
17: Annular elastic element 171: First through-hole
172: Second end surface 18: Annular element
181: Second through-hole 182: Third end surface
191: Elastic function portion 192: Tube stopper
2: Holder 2A: First joint frame
2B: Second joint frame 21: Tubular body
22: Drop prevention claw
23: Elastic function portion 24: Engaging claw
25: Engaged portion 26: Flange portion
4: Heater 41: Heat source 42: Heat transfer section
5: Extractor 51, 52, 53: Actuator
511, 521, 531: Support member 522, 532: Seal clamping member
7: Tube 9A, 9B: Connection apparatus 91: Housing
93: Handle 931: Pivot axis 932: Seal clamping member
94: Movable element 941: Operation lever
942: Seal clamping member 95: Thrust mechanism
x: First direction x1: First side in the first direction
x2: Second side in the first direction
y: Second direction

1. A tube connection system comprising:
a first fluid-path member and a second fluid-path member;
a holder that holds the first fluid-path member and the second fluid-path member;
a heater; and
an extractor, wherein
each of the first fluid-path member and the second fluid-path member includes an internal channel penetrating in a first direction, a fluid path end located at an end of the internal channel and capable of being fusion-bonded, and a sealing element having a higher thermal conductivity than that of the fluid path end and covering the fluid path end,
the first fluid-path a member includes first tube connector,
the second fluid-path member includes a second tube connector,
each of the first tube connector and the second tube connector includes a tube attachment portion disposed on a first side in the first direction, the fluid path end disposed on a second side in the first direction, and the internal channel penetrating from the tube attachment portion to the fluid path end in the first direction,
the holder includes a first joint frame that holds the first tube connector and a second joint frame that holds the second tube connector,
when the first joint frame and the second joint frame are fitted together and integrated, the first joint frame and the second joint frame are capable of holding the first tube connector and the second tube connector with the sealing elements of the first fluid-path member and the second fluid-path member facing and being pressed against each other,
at least one of the first joint frame and the second joint frame includes an elastic function portion,
the elastic function portion causes the sealing elements of the first fluid-path member and the second fluid-path member to be pressed against each other,
the heater is capable of heating the sealing element of each of the first fluid-path member and the second fluid-path member, and
the extractor is capable of pulling out the sealing element of each of the first fluid-path member and the second fluid-path member in a second direction orthogonal to the first direction.

2. The tube connection system according to claim 1, wherein the extractor is configured to fix the first joint frame and the second joint frame in a fitted position, grip the sealing elements with a seal clamping member capable of heating the sealing elements, and then pull out the sealing elements by moving the seal clamping member in the second direction.

3. The tube connection system according to claim 1, wherein the extractor is configured to grip the sealing elements with a seal clamping member capable of heating the sealing elements in a state where the first joint frame and the second joint frame are fitted together, and then pull out the sealing elements by a mechanism that pushes off at least one of the first joint frame and the second joint frame.

4. The tube connection system according to claim 1, wherein the fluid path end includes an annular first end surface facing the second side in the first direction and a fluid path end recess that is recessed from the first end surface toward the first side in the first direction on a radially inner side of the first end surface, and
the fluid path end recess surrounds the internal channel as viewed in the first direction.

5. The tube connection system according to claim 4, wherein each of the first tube connector and the second tube connector includes an annular elastic element attached to the fluid path end recess and having a higher heat resistant temperature than that of the first end surface,
the annular elastic element includes a first through-hole communicating with the internal channel and a second end surface located on the second side in the first direction from the first end surface in a natural state and facing the second side in the first direction, and
the sealing element covers the first end surface and the second end surface.

6. The tube connection system according to claim 4, wherein each of the first tube connector and the second tube connector includes an annular element attached to the fluid path end recess and having a higher heat resistant temperature than that of the first end surface,
the annular element includes a second through-hole communicating with the internal channel and a third end surface facing the second side in the first direction, and
the sealing element covers the first end surface and the third end surface.

7. The tube connection system according to claims 1, wherein the sealing element includes a thin metal plate or a thin metal film.

8. The tube connection system according to claim 1, wherein the sealing element includes a first portion overlapping with the fluid path end as viewed in the first direction, and a second portion extending out from the first portion in a direction orthogonal to the first direction.

9. The tube connection system according to claim 8, wherein the second portion is provided with an anti-slip structure on a first surface thereof that faces the first side in the first direction.

10. The tube connection system according to claim 1, wherein each of the first fluid-path member and the second fluid-path member includes a tube stopper that is attachable in such a manner that a tube fitted over the tube attachment portion is interposed between the tube stopper and the tube attachment portion.

11. A tube connector set that constitutes the tube connection system as set forth in claim 1, wherein
the tube connector set includes the first fluid-path member and the second fluid-path member.
